(19) United States
(12) Patent Application Publication
(10) Pub. No.: US 2015/0119847 A1
(43) Pub. Date: Apr. 30, 2015

(54) Atraumatic Guidewire And Method Of Use

(71) Applicant: MedWerks, LLC, St. Paul, MN (US)

(72) Inventors: Robert F. Wilson, Roseville, MN (US); Uma S. Valeti, St. Paul, MN (US); John P. Gainor, Mendota Heights, MN (US)

(21) Appl. No.: 14/527,720

(22) Filed: Oct. 29, 2014

Related U.S. Application Data
(60) Provisional application No. 61/897,138, filed on Oct. 29, 2013.

(51) Int. Cl. A61M 25/09

(52) U.S. Cl. CPC A61M 25/09; A61M 2025/09008; A61M 2025/09175
USPC 604/500; 604/529

(57) ABSTRACT

An atraumatic guidewire kit and method including a guidewire having a soft tip of increased diameter that spreads any force placed on tissue over an increased area resulting in reduced trauma to the tissue.

### RELATED APPLICATIONS

This application claims priority to U.S. Provisional Application Ser. No. 61/897,138 filed Oct. 29, 2013 entitled Atraumatic Guidewire And Method Of Use, which is hereby incorporated herein by reference in its entirety.

### FIELD OF THE INVENTION

The present invention relates to the field of guidewires used in the practice of medicine. In particular, the present invention relates to guidewires used as aids to navigation of vascular anatomy and cardiac structures.

### BACKGROUND OF THE INVENTION

There are a number of medical conditions that necessitate physician intervention by catheter in order to provide diagnosis of or therapy for diseases of the vascular or cardiac systems. In many of the patients that require this type of treatment, the nature of their disease is such that their anatomy does not allow for easy or safe passage of catheters through the vasculature or to the target location. In these cases, a guidewire is often used to reach the target location followed by the catheter that tracks over that guidewire.

An additional risk to the interventional procedure involves the interaction of that guidewire with the anatomy of the patient. During the advancement or retraction of the catheter over the wire, or during the treatment procedure itself, the wire is often in contact with fragile anatomic structures such as arteries, veins or the valves or chambers of the heart.

In the particular case of transcatheter aortic valve replacement (TAVR), there is a need to place a relatively stiff guidewire across the native aortic valve in order to guide the placement of the valve delivery system. The interaction of this relatively stiff guidewire with a delivery system that can also be quite stiff results in the potential for significant application of force at the distal end of the delivery system and in particular on the distal end of the guidewire.

Guidewires commonly used for these types of procedures are necessarily quite small in diameter, as they are intended to be passed through a small diameter lumen in the delivery system or through a sheath with a small inner diameter. The guidewire sizes typically used in an interventional cardiology procedure range from 0.014″ to 0.038″ in diameter. For a TAVR implant procedure, a 0.035″ diameter guidewire is most commonly used. Due to the small size of these wires and therefore the limited surface contact that they have with the tissue at the distal end of the system, there is great potential for a high load to be transferred to fragile tissue at focal points during the use of a guidewire-based system.

To counteract some of the concerns related to the transfer of force to vulnerable tissue, guidewires are typically designed to taper in stiffness from the proximal end to the distal end, in order to reduce the risk of damage to the vasculature. There are a great number of tip configurations that have been developed to provide a combination of functional stiffness, steerability and distal softness. Generally, this is accomplished by designing a guidewire with a tapered core wire that is contained within an outer coil affixed to each end of the core wire. This provides some body and kink resistance to the wire while maintaining flexibility. While this type of design allows the guidewire to flex and distort in an attempt to manage distal displacement of the guidewire, it does not control force application.

The safety benefit of using a guidewire during an interventional cardiology procedure is well-established, both as an aid in navigating difficult anatomy and in stabilizing a catheter. However, a guidewire design has yet to be developed that substantially reduces the risk caused by translating damaging force application to tissues during guidewire use.

### SUMMARY OF THE INVENTION

According to the invention, a guidewire is disclosed that has a distal segment of substantially greater surface area than that of the body of the guidewire. The intent of the larger diameter distal segment of the guidewire is to provide greater contact surface area between the guidewire and potentially fragile tissues in order to distribute loads across that greater surface area and result in lower focal force application. This larger diameter distal segment may also be constructed of a highly compliant material such that it can act as a shock absorber to absorb displacement through deflection or compression rather than direct force translation.

In one embodiment, this guidewire may have a large-diameter distal end constructed of a foam or some other relatively soft material. This foam is preferably expanded to a desired diameter larger than the diameter of the body of the guidewire for use in the anatomy, but may be compressible in order to fit within the lumen of a standard guide catheter. This guidewire with a compressible, soft, expandable distal tip is designed to be self-expanding upon release from the constraints of a catheter. It can be inserted through a typical catheter or sheath that would be used with a 0.035″ diameter guidewire, but has a greatly increased surface area upon expansion.

In another embodiment, the guidewire may have a large-diameter distal end constructed of a foam or some other relatively soft material. This distal end is larger than the diameter of the body of the guidewire for use in the anatomy and is intended to be inserted at a larger diameter than typically used for a guidewire. This larger diameter is acceptable when the device that is ultimately passed over the guidewire is of a greater diameter as well, which requires a larger vascular introducer in order to provide the desired treatment. In this case, the guidewire of the present invention is placed as a secondary step following placement of a first guidewire. The guidewire of the present invention contains a lumen within the larger-diameter distal tip that can be used to track this guidewire over a first guidewire that has been placed in the appropriate position.

In another embodiment, the lumen used to track the first guidewire is of a monorail design that allows for a rapid exchange of devices. This monorail lumen may be coaxial with the guidewire of the device to prevent buckling of the first guidewire and to provide greater support between the first guidewire and the guidewire of this design.

In yet another embodiment, the guidewire may have a large-diameter distal end constructed of a rolled material. This rolled material is constructed in such a way that when contained within a catheter, the distal end is contained at a smaller diameter that is smaller than the expanded diameter. Upon release from the constrained diameter, the distal end expands to a larger diameter that provides a greater surface area of contact than that of the constrained diameter. This constrained diameter may be again achieved by retracting the distal end into a constraining catheter.

In another embodiment of this design, the distal end of the guidewire may be shaped into a preferred configuration related to the intended use or treatment being provided. This pre-shaped distal end may have a component made of a superelastic material such as Nitinol, or can be stainless steel or any of a number of biocompatible materials.

In yet another embodiment of this design, the distal end of the guidewire may be shapeable such that the physician operator can bend the tip into a preferred shape. As such, a component of the distal end must be malleable in order to retain the preferred shape.

In each of these embodiments, the distal tip can be envisioned to be constructed of a soft foam, but may also be built from materials such as a braided wire or polymer, a polymeric extrusion with one or more lumens in order to accommodate a core wire and/or a monorail guide port, an expandable non-oriented fiber matrix, a balloon, or any expandable or relatively compliant material that provides the same benefit of increased distal surface area in order to better distribute forces throughout the anatomy.

In each of these embodiments, the guidewire may be constructed in such a way that there is a lumen available for a shaped stylet as a steering mechanism. This stylet may be advanced or retracted as desired in order to provide a shape change to the distal end of the guidewire as may be necessary to navigate through the anatomy.

Additionally, the guidewire may be constructed in such a way that the distal end of the guidewire is coated in an anticoagulant such as heparin in order to prevent blood clot from forming on the guidewire. The distal end of the guidewire may also be constructed of or loaded with a radiopaque material in order to better visualize the expanded diameter of the wire via fluoroscopy.

In yet another embodiment of the design, the guidewire can be constructed in such a way that the distal tip of the wire is conductive with a means for transferring an electrical current through the length of the guidewire. In addition to the atraumatic benefits of the previously described guidewire design, a wire with a conductive distal end can be used as a lead for pacing a chamber of the heart.

In yet another embodiment of the design, the lumen available for the stylet may be also used for infusion of medication, contrast or other diagnostic or therapeutic means. Additionally, the lumen may be used to measure blood pressure, collect blood samples or for other diagnostic uses.

The drawings describing the function of this invention focus on the left ventricle of the heart. However, a guidewire of this design may be used for treatment in a number of anatomic locations where a less-traumatic wire placement is desired, including the left atrium, the right atrium and ventricle, the arterial and venous vasculature as well as other non-cardiovascular uses such as the digestive system, the urinary system or other areas in which passage of a guidewire is deemed necessary or helpful.

### BRIEF DESCRIPTION OF THE DRAWINGS

FIG. 1 is a plan view of an embodiment of a device of the invention;

FIG. 2 is a depiction of a distal expanded segment of an embodiment of a device of the invention sitting in the LV;

FIGS. 3a-3c are a sequence of an embodiment of a device of the invention in which a distal end of an embodiment of a guidewire is expanding upon exiting a distal end of a sheath;

FIGS. 4a-4c are a sequence of an embodiment of a device of the invention being inserted into an LV;

FIG. 5a is a cross section of an embodiment of a device of the invention in a compressed configuration; and,

FIG. 5b is a cross section of the device of FIG. 5a in an expanded configuration.

### DETAILED DESCRIPTION OF THE DRAWINGS

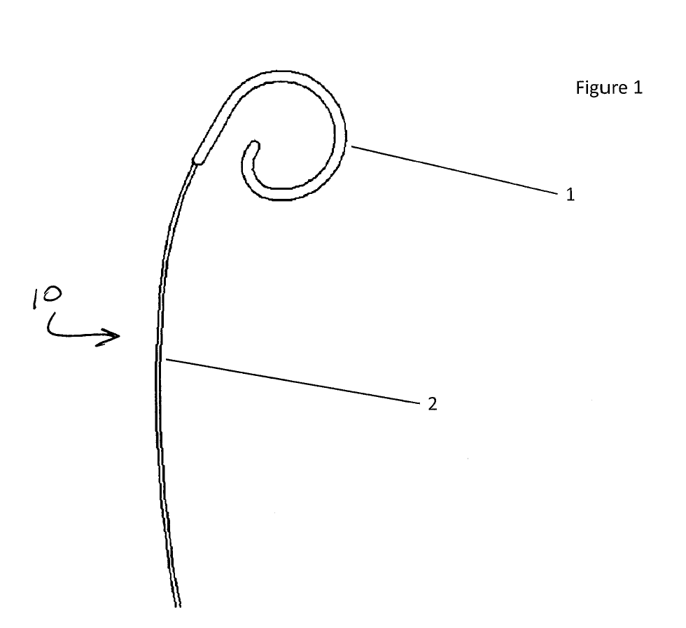

Referring now to the figures, and first to FIG. 1, there is shown an embodiment of a guidewire 10 of the present invention with a larger diameter tip portion 1 and a guidewire body 2. This larger diameter tip 1 may be manufactured from a compressible and expandable material such as a foam, polymeric or metallic mesh cylinder, a braid, gel, balloon or the like. The larger diameter tip 1 of the guidewire 10 may be manufactured with a pre-curved shape intended for a particular anatomic application, or the tip 1 may be provided in a configuration that is shapeable by the user into a desired configuration. FIG. 1 depicts a possible shape of the larger diameter tip 1 suited for a left ventricular application.

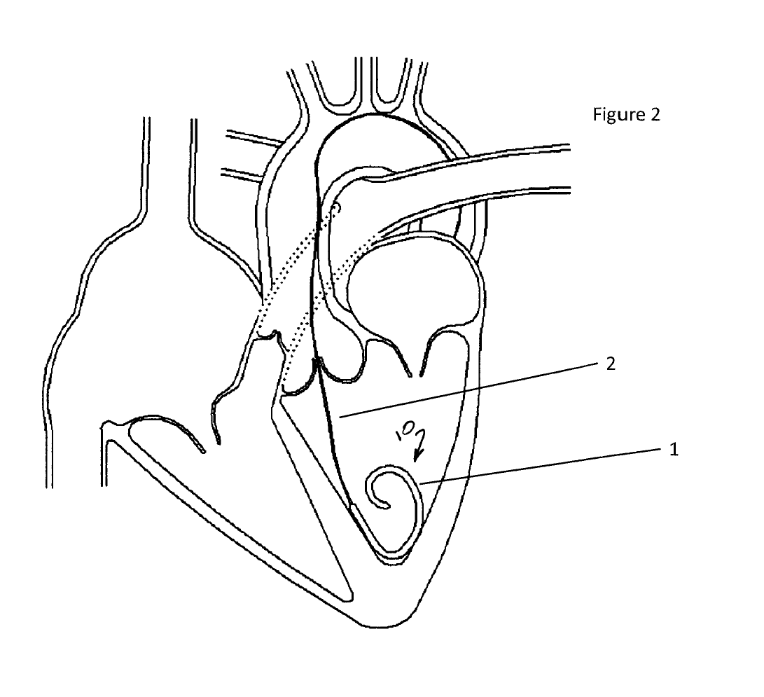

FIG. 2 depicts guidewire 10 in a left ventricular application. The curve of the larger diameter tip 1 is intended to sit within the left ventricular apex to provide protection to the ventricular wall either through a distribution of force across a greater wire surface area or as a shock absorber, intended to absorb axial guidewire displacement of the guidewire body 2 through deformation of the larger diameter tip 1.

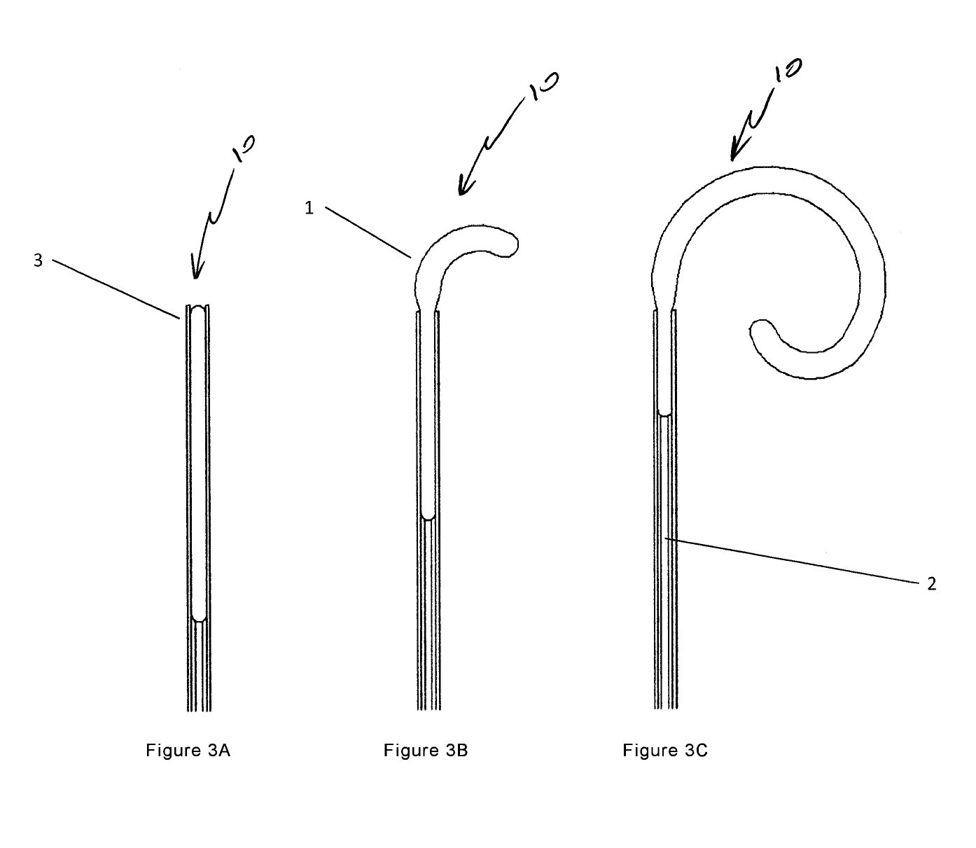

FIGS. 3a-3c depicts a guidewire 10 of the invention reversibly compressed for insertion into the vasculature. Beginning with FIG. 3a, the guidewire 10 has been compressed into the introducer sheath 3. The introducer sheath 3 constrains the larger diameter tip 1 during introduction into the vasculature. Upon advancement of the guidewire body 2, best seen in FIG. 3b, the larger diameter tip 1 exits the introducer sheath 3 and expands to a predetermined diameter. As seen in FIG. 3c, as the tip 1 emerges, it both expands and resumes the intended guidewire shape as pre-manufactured or as curved by the user at the point of use.

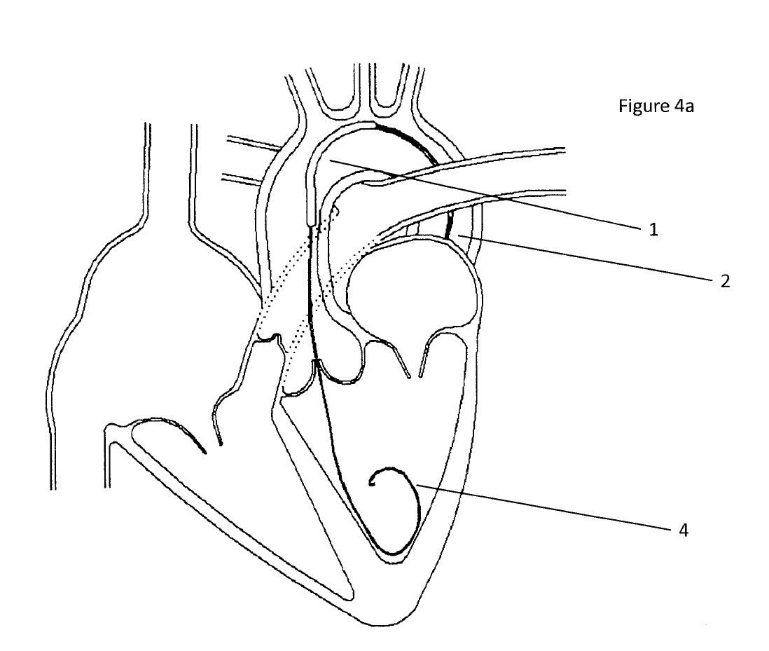

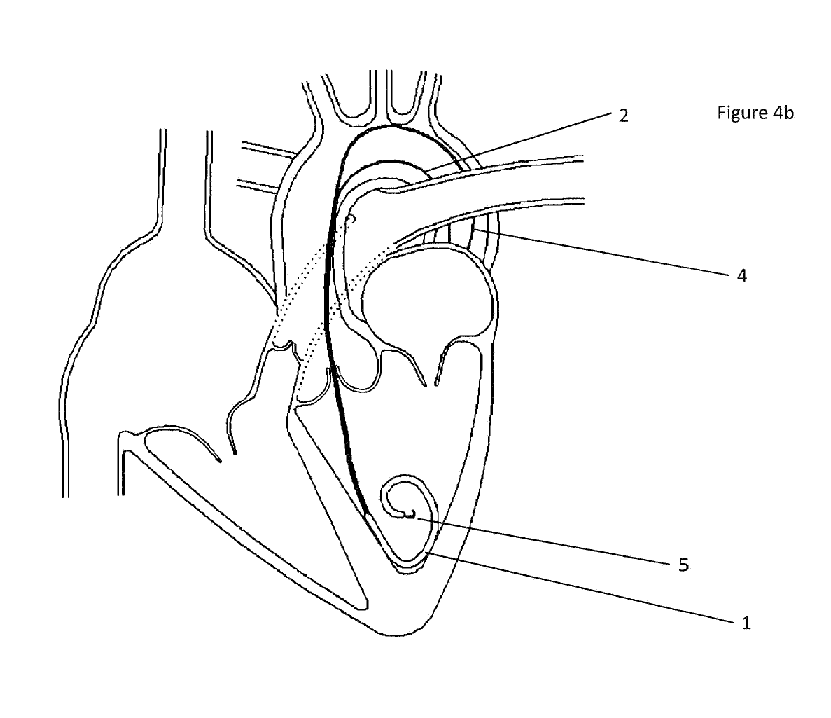

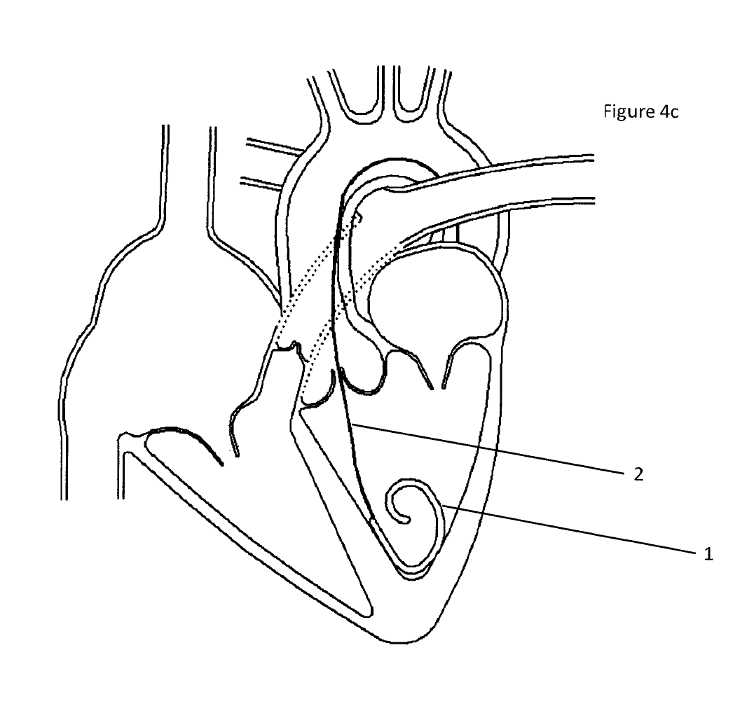

FIGS. 4a-4c depict a guidewire 10 of the invention being advanced to the left ventricle (LV) of a patient over a secondary guidewire 4. First, as shown in FIG. 4a, the secondary guidewire 4 is placed in a preferred position in the LV. The proximal end (not shown) of the secondary guidewire 4 is inserted into a distal end of a lumen of the larger diameter tip 1 and the proximal end of the secondary guidewire 4 is advanced proximally until it exits the proximal end of the larger diameter tip 1. This is done without disrupting the position of the distal end of the secondary guidewire 4. As shown in FIGS. 4b and 4c, the guidewire body 2 is advanced in parallel over the secondary guidewire 4 while the position of the secondary guidewire 4 is held stationary. In this manner, the larger diameter tip 1 of the guidewire tracks over the secondary guidewire 4.

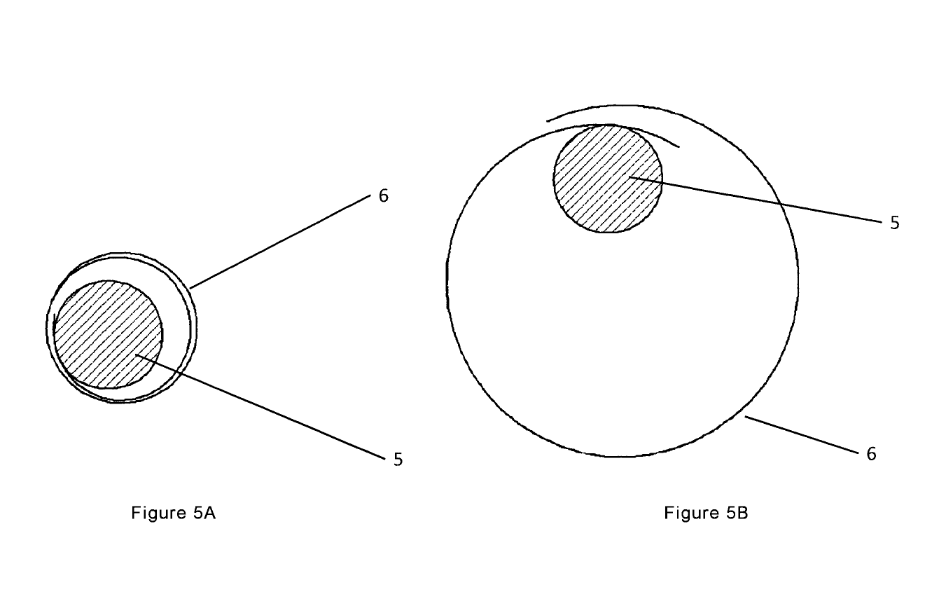

FIGS. 5a and 5b depict how the larger diameter tip 1 of the guidewire 10 is positioned relative to the distal end of the secondary guidewire 4. Due to the nature of the lumen constraining the guidewire 10 of the present invention to the secondary guidewire 4, the body of the secondary guidewire 4 and the guidewire body 2 of the present invention do not necessarily maintain a coaxial position throughout the wire lengths.

FIG. 5b describes the position of the larger diameter tip 1 and the guidewire body 2 after removal of the secondary guidewire 4.

FIG. 5c is a cross-sectional view that describes another embodiment of the current invention in which the larger diameter tip 1 of the wire may be constructed from a rolled film with a predetermined expanded shape. This rolled film 6 is attached to a core wire 5 that is in turn a component of the guidewire body 2. In an unconstrained position, the rolled film 6 expands to create a larger-diameter cylindrical shape with an increased surface area. When circumferentially constrained, the rolled film 6 can be compressed around the core wire in such a way that it can be packed in to a much smaller introducer sheath. In one embodiment of this invention, the compressed diameter of the rolled distal tip is no larger than the guidewire body 2 such that the introducer size required for the guidewire 10 is minimized.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

We claim:

1. A guidewire comprising:
a body having a diameter;
a distal tip portion having a diameter greater than the body diameter.

2. The guidewire of claim 1 wherein said distal tip portion is compressible.

3. The guidewire of claim 1 wherein said distal tip portion is expandable.

4. The guidewire of claim 1 wherein said distal tip portion comprises a material selected from the group foam, polymeric mesh, metallic mesh, braid, gel and balloon.

5. The guidewire of claim 1 wherein said distal tip portion is shapeable.

6. The guidewire of claim 1 wherein said distal tip portion is curved.

7. The guidewire of claim 1 further comprising a lumen passing through said body and said distal tip portion.

8. A guidewire kit comprising:
an atraumatic guidewire comprising:
a body having a diameter;
a distal tip portion having a diameter greater than the body diameter; and,
a lumen extending through said body and said distal tip portion;
a secondary guidewire sized to fit through said lumen; and,
a sheath sized to slidingly contain said atraumatic guidewire such that said distal tip portion is in a compressed state when disposed within said sheath.

9. The kit of claim 8 wherein said distal tip portion is expands when released from said sheath.

10. The guidewire of claim 8 wherein said distal tip portion comprises a material selected from the group foam, polymeric mesh, metallic mesh, braid, gel and balloon.

11. The guidewire of claim 8 wherein said distal tip portion is shapeable.

12. The guidewire of claim 8 wherein said distal tip portion is curved.

13. A method of atraumatically placing a guidewire at a target location comprising:
providing a distal tip portion on the guidewire having a feature that results in increased surface area contacting tissue of the target location; and,
navigating the guidewire to the target location.

14. The method of claim 13 wherein providing a distal tip portion on the guidewire having a feature that results in increased surface area contacting tissue of the target location comprises providing a distal tip portion on the guidewire that has a diameter greater than a diameter of a body of the guidewire.

15. The method of claim 13 wherein providing a distal tip portion on the guidewire having a feature that results in increased surface area contacting tissue of the target location comprises providing a distal tip portion of a material selected from the group foam, polymeric mesh, metallic mesh, braid, gel and balloon.

16. The method of claim 13 wherein providing a distal tip portion on the guidewire having a feature that results in increased surface area contacting tissue of the target location comprises providing a distal tip portion that is compressible and expandable.

17. The method of claim 13 wherein navigating the guidewire to the target location comprises:
navigating a secondary guidewire to the target location;
providing a lumen in the guidewire;
feeding the guidewire over the secondary guidewire using the lumen.

18. The method of claim 13 further comprising: releasing the guidewire from a sheath, thereby allowing the distal tip portion to expand.

19. The method of claim 13 wherein providing a distal tip portion on the guidewire having a feature that results in increased surface area contacting tissue of the target location comprises providing a distal tip portion having a curve.

20. The method of claim 13 further comprising releasing the guidewire from a sheath, thereby allowing the distal tip portion to resume a preformed curvature.
